(19) United States
(12) Patent Application Publication
(10) Pub. No.: US 2005/0203062 A1
(43) Pub. Date: Sep. 15, 2005

(54) Use of tagatose in laxatives

(75) Inventor: Gilbert V Levin, Annapolis, MD (US)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: Spherix Incorporated, Beltsville, MD (US)

(21) Appl. No.: 10/508,431

(22) PCT Filed: Mar. 20, 2003
(86) PCT No.: PCT/US03/08445

§ 371 Date: May 31, 2005

Related U.S. Application Data
(60) Provisional application No. 60/365,793, filed on Mar. 21, 2002.

(51) Int. Cl. A61K 31/715

(52) U.S. Cl. 514/61

(57) ABSTRACT

Tagatose is substituted for the sugars, sweeteners and/or bulking agents presently used in fiber laxatives either alone or in combination with other sweeteners and/or bulking agents.

### BACKGROUND OF THE INVENTION

This invention relates to the use of tagatose in laxatives, particularly fiber laxatives.

U.S. Pat. No. 4,786,722 discloses edible formulations and methods for preparation of edible formulations in which D-tagatose is used as a low calorie carbohydrate sweetener and bulking agent. In this patent, D-tagatose is described as useful in food stuffs and other edible formulations for people whose metabolizable carbohydrate intake must be restricted because of conditions such as diabetes mellitus or obesity.

Fiber laxatives are used to treat constipation and to restore bowel regularity. Such laxatives are frequently advertised as 100 percent natural psyllium husks, and are sold as a dietary fiber supplement. However, in order to overcome the unpleasant taste of the active ingredient, psyllium husks, additives are required. Typically, these additives are dextrose and citric acid, but other sweeteners may be used in place thereof, or in conjunction with dextrose, such as sucrose, fructose and one or more of the sugar alcohols or “polyols,” or high intensity sweeteners may be used. The latter, however, cannot serve alone because of the added bulk required from the sweetener in the product.

The label on the generic fiber laxative described above recommends that the product be taken three times daily. Each recommended dose is seven grams, of which three grams, some 43 percent of the total dose, are the sugar dextrose. This amount approximates the 3.4 grams of psyllium husk, the active agent, in each dose according to the label. Thus, nine grams of sugar are consumed daily, providing 36 calories, 48 percent of the total daily caloric intake of 75 calories per the product label.

### SUMMARY OF THE INVENTION

In accordance with this invention, tagatose is substituted for all or part of the sugars or sweeteners presently used in fiber laxatives. The fiber laxatives may be of the psyllium variety or may be based on alternative fiber sources. There is thus provided a laxative containing tagatose as a sweetening agent alone, or with other full- or low-caloric sweetening agents or bulking agents, in an amount sufficient to sweeten said laxative.

### DETAILED DESCRIPTION OF THE INVENTION

This invention provides a process for the preparation of a laxative which comprises using tagatose as all or part of the sweetening agent. The use of tagatose as a substitute for sweeteners presently used provides the following advantages:

1. Reduction in caloric intake.

Tagatose has been approved by the US Food and Drug Administration at a caloric value of 1.5 calories per gram. Research shows that the true value may be considerably lower, i.e., as low as 0.15 calories per gram. However, even at the upper value of 1.5, the use of tagatose as the total sweetener will reduce the daily caloric intake of a person using a fiber laxative by 9×1.5×100/36=62.5 percent. Corresponding caloric reductions would occur were tagatose used with other sweeteners. Its use with low-calorie, high-intensity sweeteners would still provide up to the total 62.5 percent caloric reduction. Thus, tagatose provides a significant reduction in caloric intake for the present population of which nearly two-thirds are obese, particularly the aging adults, who are also the primary consumers of fiber laxatives.

2. No rise in blood glucose.

Unlike the sugars and sweeteners currently used in fiber laxatives, tagatose does not produce a rise in blood glucose. Indeed, it has been determined that tagatose blunts the rise in blood glucose from subsequent intake of glucose-producing foods.

3. Use by diabetics.

Because tagatose produces no rise in blood glucose, its use in fiber laxatives makes these products safe for use by diabetics. In addition, tagatose has been shown in clinical trials to be an effective treatment for Type 2 diabetes.

4. Enhancement of laxative effect.

Tagatose taken in amounts approaching 30 or 40 grams per day is known to have a Taxation effect. The prescribed dose of nine grams per day in the fiber laxatives will aid in the laxative effect of the psyllium husk. The proportions of tagatose and psyllium husk may be varied for different degrees of Taxation. More than one composition of product may be desirable to fit the range of population requirements.

5. Conversion to OTC product.

The use of tagatose in fiber laxatives can convert such dietary supplements into over-the counter (OTC) drugs for the more secure and effective treatment of constipation. In addition to the relief of constipation, other demonstrated health claims can be made. These include anti-cariogenic, anti-plaque, and, to the extent the product is retained in the mouth, tooth-whitening.

6. Use alone or in combination.

Because of its excellent taste, tagatose may be used as the sole sweetener in fiber laxatives, or may be used in conjunction with other full-bulk sweeteners or with high intensity sweeteners.

1. A process for the preparation of a laxative which comprises using tagatose as a component.

2. A process as defined in claim 1 in which the tagatose serves as all or part of the sweetening agent of the laxative.

3. A process as defined in claim 1 in which the tagatose serves as all or part of the bulking agent.

4. A process as defined in claim 1 in which tagatose is combined with other sweetening agent(s).

5. A process as defined in claim 1 in which tagatose is combined with other bulking agent(s).

6. A process as defined in claim 1 in which tagatose is used in an amount to supply all or part of the sweetening.

7. A process as defined in claim 1 in which tagatose is used in an amount to supply all or part of the bulk.

8. A process as defined in claim 1 wherein the laxative is a fiber laxative.

9. A fiber laxative containing tagatose as a sweetening agent in an amount sufficient to sweeten said laxative either used alone or in combination with other sweeteners.

10. A fiber laxative containing tagatose as a bulking agent in an amount sufficient to supply the required bulk of the laxative either used alone or in combination with other bulking agents.

11. A laxative in which tagatose is used to supply both bulk and sweetness by itself or in combination with other sweetening and/or bulking agents.

12. A fiber laxative in which tagatose is used to supply both bulk and sweetness by itself or in combination with other sweetening and/or bulking agents.
